(19) United States
(12) Patent Application Publication
(10) Pub. No.: US 2008/0014596 A1
(43) Pub. Date: Jan. 17, 2008

(54) ADDL Binding to Hippocampal Neurons

(76) Inventors: Jasna Jerecic, San Francisco, CA (US); Grant A. Krafft, Glenview, IL (US)

Correspondence Address: Jane Massey Licata, Esquire;Licata & Tyrrell P.C., 66 E. Main Street, Marlton, NJ, 08053, US

(21) Appl. No.: 11/560,834

(22) Filed: Nov. 16, 2006

Related U.S. Application Data
(60) Provisional application No. 60/737,518, filed on Nov. 16, 2005.

(51) Int. Cl. G01N 33/567; G01N 33/53

(52) U.S. Cl. 435/7.2

(57) ABSTRACT

Disclosed herein are methods for the quantification of ADDL binding to neuronal cells, including, but not limited to, primary cultures of hippocampal neurons. The method identifies and selects neurons based on any means capable of distinguishing neuronal cells, including, but not limited to, MAP2 immunoreactivity, which ensures that glial cells are excluded from an ADDL binding analysis; antibodies selective for neuronal cell surface receptors and/or other surface markers; reagents specific for neuronal signalling markers present intracellularly; and the like. Furthermore, ADDL binding occurs in a sub-population of 16 DIV neurons and is heterogeneous in intensity among individual cells. Also, ADDL binding can be further specified and quantified by using additional markers. Additionally, the presence or absence of ADDL binding is used to identify, characterize, analyze, assess, and/or evaluate agents (e.g., including, but not limited to, small molecules, antibodies, chemical compounds, dietary components, environmental conditions, etc.) that modulate ADDL binding. Such modulation can be positive or negative, including, but not limited to, ADDL binding inhibition, either total inhibition or partial inhibition, and the like.

### CROSS-REFERENCE TO RELATED APPLICATIONS

This application claims priority from U.S. Patent App. No. 60/737,518 filed Nov. 16, 2005.

### BACKGROUND

1. Field

The invention disclosed herein concerns the fields of biology, medicine, and the like. In particular, the invention concerns Alzheimer's disease, Down's syndrome, mild cognitive impairment, and the like. Specifically, the invention concerns the assay, analysis, and characterization of soluble amyloid beta oligomers, including the assay, analysis, and characterization of inhibitors of soluble amyloid beta oligomer assembly and activity.

2. Related Art

Alzheimer's disease (AD) is a progressive and degenerative dementia (Terry, R. D. et al. (1991) “Physical basis of cognitive alterations in Alzheimer's disease: synapse loss is the major correlate of cognitive impairment” Ann. Neurol., vol. 30, no. 4, pp. 572-580; Coyle, J. T. (1987) “Alzheimer's Disease” in Encyclopedia of Neuroscience, Ed. G. Adelman, pp 29-31, Birkhäuser: Boston-Basel-Stuttgart). In its early stages, however, AD manifests primarily as a profound inability to form new memories (Selkoe, D. J. (2002) “Alzheimer's disease is a synaptic failure” Science, vol. 298, pp. 789-791). The basis for this specific impact is not known, but evidence favors involvement of neurotoxins derived from amyloid beta (Aβ). Aβ is an amphipathic peptide whose abundance is increased by mutations and risk factors linked to AD. Fibrils formed from Aβ constitute the cores of amyloid plaques, which are hallmarks of AD brain. Analogous fibrils generated in vitro are lethal to cultured brain neurons. These findings provided the central rationale for the original amyloid cascade hypothesis, a remarkably productive theory in which memory loss was proposed to be the consequence of neuron death caused by fibrillar Aβ.

Despite its strong experimental support and intuitive appeal, the original amyloid cascade hypothesis has proven inconsistent with key observations, including the poor correlation between dementia and amyloid plaque burden (Katzman, R. (1988) “Clinical, pathological, and neurochemical changes in dementia: a subgroup with preserved mental status and numerous neocortical plaques” Ann. Neurol., vol. 23, no. 2, pp. 138-144). Particularly telling are recent studies of experimental AD vaccines done with transgenic hAPP mice (Dodart, J. C. et al. (2002) “Immunization reverses memory deficits without reducing brain Abeta burden in Alzheimer's disease model” Nat. Neurosci., vol. 5, pp. 452-457; Kotilinek, L. A. et al. (2002) “Reversible memory loss in a mouse transgenic model of Alzheimer's disease” J. Neurosci., vol. 22, pp. 6331-6335). These mice provide good models of early AD, developing age-dependent amyloid plaques and, most importantly, age-dependent memory dysfunction. Two surprising findings were obtained when mice were treated with monoclonal antibodies against Aβ: (1) vaccinated mice showed reversal of memory loss, with recovery evident in 24 hours; (2) cognitive benefits of vaccination accrued despite no change in plaque levels. Such findings are not consistent with a mechanism for memory loss dependent on neuron death caused by amyloid fibrils.

Salient flaws in the original hypothesis have been eliminated by an updated amyloid cascade that incorporates a role for additional neurologically active molecules formed by Aβ self-assembly. These molecules are soluble Aβ oligomers. Oligomers are metastable and form at low concentrations of Aβ1-42 (Lambert, M. P. et al. (1998) “Diffusible, nonfibrillar ligands derived from Abeta 1-42 are potent central nervous system neurotoxins” Proc. Natl. Acad. Sci. USA, vol. 95, pp. 6448-6453). Essentially the missing links in the original cascade, Aβ oligomers rapidly inhibit long-term potentiation (LTP), a classic experimental paradigm for memory and synaptic plasticity. In the updated cascade: (1) memory loss stems from synapse failure, prior to neuron death; and (2) synapse failure is caused by Aβ oligomers, not fibrils (Hardy, J. & Selkoe, D. J. (2002) “The amyloid hypothesis of Alzheimer's disease: progress and problems on the road to therapeutics” Science, vol. 297, pp. 353-356). Recent reports show soluble oligomers occur in brain tissue and are strikingly elevated in AD (Kayed, R. et al. (2003) “Common structure of soluble amyloid oligomers implies common mechanism of pathogenesis” Science, vol. 300, pp. 486-489; Gong, Y. et al. (2003) “Alzheimer's disease-affected brain: presence of oligomeric Aβ ligands (ADDLs) suggests a molecular basis for reversible memory loss” Proc. Natl. Acad. Sci. USA, vol. 100, pp. 10417-10422) and in hAPP transgenic mice AD models (Kotilinek, L. A. et al. (2002) “Reversible memory loss in a mouse transgenic model of Alzheimer's disease” J. Neurosci., vol. 22, pp. 6331-6335; Chang, L. et al. (2003) “Femtomole immunodetection of synthetic and endogenous amyloid-β oligomers and its application to Alzheimer's Disease drug candidate screening” J. Mol. Neurosci., vol. 20, pp. 305-313).

Amyloid beta immunotherapy for Alzheimer's disease has shown initial success in mouse models of AD and in human patients not susceptible to meningoencephalitis. Disclosed herein are monoclonal antibodies against soluble Aβ oligomers (ADDLs). The antibodies distinguish between AD and control human brain extracts. The antibodies identify endogenous oligomers in AD brain slices and also bind to cultured hippocampal cells. The antibodies neutralize endogenous and “synthetic” ADDLs in solution. So-called “synthetic” ADDLs are produced in vitro by mixing purified amyloid β 1-42 under conditions that produce ADDLs, see U.S. Pat. No. 6,218,506. One of the antibodies, 20C2, shows high selectivity for 3-24mers, but minimal detection of monomer Aβ peptides. Recognition of ADDLs by 20C2 is not blocked by short peptides that encompass the linear sequence of Aβ 1-42 or by Aβ 1-40. However, binding is blocked by Aβ 1-28, suggesting an epitope based on conformationally unique structures also attained with Aβ 1-28.

AD is a fatal progressive dementia that has no cure at present. Although the molecular basis of the disease is not established, considerable evidence indicates that it is a proteinopathy involving neurotoxins derived from the 42-amino acid peptide amyloid beta (Aβ). A recent revision of the major “amyloid cascade hypothesis” to explain disease progression states that small soluble Aβ oligomers, as well as the larger Aβ fibrils that constitute the core of plaques, are pathogenic (Hardy, J. & Selkoe, D. J. (2002) “The amyloid hypothesis of Alzheimer's disease: progress and problems on the road to therapeutics” Science, vol. 297, pp. 353-356).

Recent studies have shown that small soluble Aβ oligomers (also called Aβ-derived diffusible ligands or ADDLs) are present in AD brain, increasing up to 70-fold over control subjects (Gong, Y. et al. (2003) “Alzheimer's disease-affected brain: Presence of oligomeric Aβ ligands (ADDLs) suggests a molecular basis for reversible memory loss” Proc. Natl. Acad. Sci. USA, vol. 100, pp. 10417-10422). The very abundance of ADDLs in AD brain suggests their potential for therapeutic drugs or vaccines. Earlier clinical trials of a vaccine have revealed that persons mounting a vigorous immune response to the vaccine exhibited cognitive benefit (Hock, C. et al. (2003) “Antibodies against beta-amyloid slow cognitive decline in Alzheimer's disease” Neuron, vol. 38, pp. 547-554). These findings indicate genuine therapeutic promise, despite the unacceptable frequency of CNS inflammation that caused early termination of part of the trial (Birmingham, K. & Frantz, S. (2002) “Set back to Alzheimer vaccine studies” Nat. Med., vol. 8, pp. 199-200).

An alternative to a live vaccine is the development of therapeutic antibodies that target ADDLs without binding monomers or fibrils (Klein, W. L. (2002) “Aβ toxicity in Alzheimer's disease: globular oligomers (ADDLs) as new vaccine and drug targets” Neurochem. Int., vol. 41, pp. 345-352). Previous work has shown that ADDLs are excellent antigens, generating oligomer-selective polyclonal antibodies in rabbits at the very low antigen concentration of ˜50 ug/ml (Lambert, M. P. et al. (2001) “Vaccination with soluble Abeta oligomers generates toxicity-neutralizing antibodies” J. Neurochem., vol. 79, pp. 595-605). Results from tg-mice models also suggest that antibodies can be successful in reversing memory decline (Dodart, J. C. et al. (2002) “Immunization reverses memory deficits without reducing brain Abeta burden in Alzheimer's disease” Nat. Neurosci., vol. 5, pp. 452-457).

Immunization of tg mice models of AD with fibrillar amyloid beta protein (Aβ) results in reduction of Aβ deposits in the brain and prevents the formation of this pathology when administered before its formation (Schenk, D. (2002) Amyloid-beta immunotherapy for Alzheimer's disease: the end of the beginning. Nat. Rev. Neurosci. 3(10):824-8; Schenk, D. et al. (1999) Immunization with amyloid-beta attenuates Alzheimer-disease-like pathology in the PDAPP mouse. Nature 400(6740): 173-7). Learning and memory deficits produced in these mice are also reduced or prevented by similar active vaccination with preparations containing fibrillar Aβ (Janus, C. et al. (2000) A beta peptide immunization reduces behavioural impairment and plaques in a model of Alzheimer's disease. Nature 408(6815):979-82; Morgan, D. et al. (2000) A beta peptide vaccination prevents memory loss in an animal model of Alzheimer's disease. Nature 408(6815):982-5). Based on results from animal models, clinical trials were initiated and showed few adverse reactions in Phase 1. However, Phase 2 trials were halted when 6% of the patients developed meningoencephalitis (Birmingham, K. & Frantz, S. (2002) Set back to Alzheimer vaccine studies. Nat. Med. 8(3):199-200; Hock, C. et al. (2003) Antibodies against beta-amyloid slow cognitive decline in Alzheimer's disease. Neuron 38(4):547-54; Orgogozo, J. M. et al. (2003) Subacute meningoencephalitis in a subset of patients with AD after Abeta42 immunization. Neurology 61(1):46-54; Schenk, D. (2002) Amyloid-beta immunotherapy for Alzheimer's disease: the end of the beginning. Nat. Rev. Neurosci. 3(10):824-8; Schenk, D. et al. (2004) Current progress in beta-amyloid immunotherapy. Curr. Opin. Immunol. 16(5):599-606). Reports of the clinical outcome of these trials revealed that after 1 year patients producing antibodies that targeted plaques had a slower rate of cognitive decline than those patients that did not produce antibodies (Hock, C. et al. (2003) Antibodies against beta-amyloid slow cognitive decline in Alzheimer's disease. Neuron 38(4):547-54). Post mortem results on two patients showed absent or sparse plaques in the neocortex, with reactive microglia suggesting an effective immune response (Ferrer, I. et al. (2004) Neuropathology and pathogenesis of encephalitis following amyloid-beta immunization in Alzheimer's disease. Brain Pathol 14(1):11-20; Nicoll, J. A. et al. (2003) Neuropathology of human Alzheimer disease after immunization with amyloid-beta peptide: a case report. Nat. Med. 9(4):448-52).

Alternative approaches to avoid inflammatory responses through the use of therapeutic antibodies are now under development (Agadjanyan, M. G. et al. (2005) Prototype Alzheimer's disease vaccine using the immunodominant B cell epitope from beta-amyloid and promiscuous T cell epitope pan HLA DR-binding peptide. J. Immunol. 174(3): 1580-6; Gelinas, D. S. et al. (2004) Immunotherapy for Alzheimer's disease. Proc. Natl. Acad. Sci. USA 101(Suppl 2):14657-62; Morgan, D. & Gitter, B. D. (2004) Evidence supporting a role for anti-Abeta antibodies in the treatment of Alzheimer's disease. Neurobiol. Aging 25(5):605-8; Schenk, D. et al. (2004) Current progress in beta-amyloid immunotherapy. Curr. Opin. Immunol. 16(5):599-606). It has been established that injections with Aβ-generated monoclonal antibodies produce cognitive improvement in tg mice models of AD. Using an antibody whose epitope targets the center of the Aβ peptide, it was shown that memory deficits can be reversed in PDAPP mice within 24 hours after treatment (Dodart, J. C. et al. (2002) Immunization reverses memory deficits without reducing brain A beta burden in Alzheimer's disease model. Nature Neuroscience 5(5):452-7). Similarly, in Tg2576 mice, memory loss was reversed using an antibody targeting the N-terminus of Aβ (Kotilinek, L. A. et al. (2002) Reversible memory loss in a mouse transgenic model of Alzheimer's disease. J. Neurosci. 22(15):6331-5).

Passive vaccination previously was shown to clear plaques from PDAPP and other tg mice models (Bacskai, B. J. et al. (2002) Non-Fc-mediated mechanisms are involved in clearance of amyloid-beta in vivo by immunotherapy. J. Neurosci. 22(18):7873-8; Bard, F. et al. (2003) Epitope and isotype specificities of antibodies to beta-amyloid peptide for protection against Alzheimer's disease-like neuropathology. Proc. Natl. Acad. Sci. USA 100(4):2023-8; Bard, F. et al. (2000) Peripherally administered antibodies against amyloid beta-peptide enter the central nervous system and reduce pathology in a mouse model of Alzheimer disease. Nat. Med. 6(8):916-9; McLaurin, J. et al. (2002) Therapeutically effective antibodies against amyloid-beta peptide target amyloid-beta residues 4-10 and inhibit cytotoxicity and fibrillogenesis. Nature Medicine 8(11): 1263-9). However, in the studies showing recovery from memory deficits, Aβ plaque burden was not decreased. A likely explanation for cognitive improvement without change in plaque burden is that these therapeutic antibodies immunoneutralize small, soluble oligomers of Aβ, which have been implicated in AD synapse failure (Lacor, P. N. et al. (2004) Synaptic targeting by Alzheimer's-related amyloid beta oligomers. J. Neurosci. 24(45): 10191-200). Aβ oligomers form at low doses of Aβ 1-42, block LTP, and specifically attach to synaptic terminals (Lacor, P. N. et al. (2004) Synaptic targeting by Alzheimer's-related amyloid beta oligomers. J. Neurosci. 24(45):10191-200; Lambert, M. P. et al. (1998) Diffusible, nonfibrillar ligands derived from Abeta1-42 are potent central nervous system neurotoxins. Proc. Natl. Acad. Sci. USA 95(11):6448-53; Wang, H. W. et al. (2002) Soluble oligomers of beta amyloid (1-42) inhibit long-term potentiation, but not long-term depression, in rat dentate gyrus. Brain Res. 924(2): 133-40; Wang, Q. et al. (2004) Block of long-term potentiation by naturally secreted and synthetic amyloid beta-peptide in hippocampal slices is mediated via activation of the kinases c-Jun N-terminal kinase, cyclin-dependent kinase 5, and p38 mitogen-activated protein kinase as well as metabotropic glutamate receptor type 5. J. Neurosci. 24(13):3370-8). These oligomers (referred to as ADDLs) are elevated in AD brain and CSF and in tg mouse models (Chang, L. et al. (2003) Femtomole immunodetection of synthetic and endogenous amyloid-beta oligomers and its application to Alzheimer's disease drug candidate screening. J. Mol. Neurosci. 20(3):305-13; Georganopoulou, D. G. et al. (2005) Nanoparticle-based detection in cerebral spinal fluid of a soluble pathogenic biomarker for Alzheimer's disease. Proc. Natl. Acad. Sci. USA 102(7):2273-76; Gong, Y. et al. Alzheimer's disease-affected brain: presence of oligomeric A beta ligands (ADDLs) suggests a molecular basis for reversible memory loss. Proc. Natl. Acad. Sci. USA 2003 100(18):10417-22).

Given these considerations, soluble amyloid beta oligomers (including ADDLs) provide an optimum target for prophylactic and/or therapeutic treatment of Alzheimer's disease, Down's syndrome, mild cognitive impairment, and the like. The present invention addresses the need to assay, analyze, and characterize soluble amyloid beta oligomers (including ADDLs), including the need to identify, assay, analyze, and characterize inhibitors of the assembly and/or activity of these oligomers.

### BRIEF SUMMARY

The embodiments disclosed herein describe methods for the quantification of ADDL binding to neuronal cells to which ADDLs bind, including, but not limited to, primary cultures of hippocampal neurons and the like. The method identifies and selects neurons based on any means capable of distinguishing neuronal cells, including, but not limited to MAP2 immunoreactivity, which ensures that glial cells are excluded from an ADDL binding analysis; antibodies selective for neuronal cell surface receptors and/or other surface markers; reagents specific for neuronal signalling markers present intracellularly; and the like. Furthermore, ADDL binding occurs in a sub-population of 16 DIV neurons and is heterogeneous in intensity among individual cells. Also, ADDL binding can be further specified and quantified by using additional markers. Additionally, the presence or absence of ADDL binding is used to identify, characterize, analyze, assess, and/or evaluate agents (e.g., including, but not limited to, small molecules, antibodies, chemical compounds, dietary components, environmental conditions, etc.) that modulate ADDL binding. Such modulation can be positive or negative, including, but not limited to, ADDL binding inhibition, either total inhibition or partial inhibition, and the like.

Also disclosed herein are methods of screening for ADDL binding inhibitors; such methods comprising the steps of adding agents suspected of being ADDL binding inhibitors to the assays disclosed herein and assessing, as disclosed herein, the effect such agents have on the binding of ADDLs to neuronal cells.

Additional embodiments comprise methods of screening for ADDL signalling, such methods comprising the steps of adding agents suspected of affecting ADDL signalling to the assays disclosed herein and assessing, as disclosed herein, the effect such agents have on ADDL signalling in neuronal cells.

Furthermore, the embodiments disclosed herein comprise methods of screening for cell types and receptors involved in ADDL signalling; such methods comprising the steps of adding agents suspected of affecting ADDL binding to particular cell types and/or receptors to the assays disclosed herein and assessing, as disclosed herein, the effect such agents have on the binding of ADDLs to particular cell types and/or receptors.

### BRIEF DESCRIPTION OF THE DRAWINGS

FIG. 1 depicts the selection of ADDL positive neurons. Total number of objects in each field was identified using DAPI nuclear stain (left panel). Neurons were visualized using the dendritic marker MAP2 (middle panel). Blue circles represent selected objects positive for the neuronal marker MAP2. Orange circles represent MAP2 negative cells excluded in downstream image processing and quantification. ADDLs were visualized using the anti-oligomer specific antibody 2B4 (right panel). ADDL binding intensities in individual neurons was measured in soma (red circles) and proximal dendrites (green).

FIGS. 2A & 2B depict single cell binding intensities in ADDL treated cells. Histograms of average dendritic intensities measured by the ArrayScan® HCS Reader in cultured hippocampal neurons. The graphs show the distribution of intensities in single cells taken from 3 wells each (approximately 800 cells/well). The histograms were fitted by two Gaussian distributions. Dashed lines indicate the individual Gaussian, and the sum is shown by a solid line. Note the ADDL concentration-dependent shift to the left of the average Ringspot binding intensities. The images on the right show representative pictures of ADDL binding at the indicated concentrations (left panels) and areas of the image measured for binding intensity quantification are outlined in green (right panels). Note that incubation with the primary ADDL-specific antibody yields a single distribution, representative of non-specific binding activity.

FIG. 3 depicts selective ADDL binding to hippocampal neurons. Image shows superimposed staining of nuclei (blue), neurons (red) and ADDLs (green). Arrows indicate binding of ADDLs to a subset of MAP2 positive neurons (2) versus (3). Note that glia, represented by nuclear stain (1) do not exhibit ADDL staining.

FIG. 4 depicts FIG. 4: Co-localization of ADDL binding to neuronal cells. Double-labeling of neuronal cultures with MAP 2 antibody (A) and anti ADDL antibody (B). Co-localization (C) shows overlap of ADDL binding cells (green) and MAP 2 positive cells (red). Nuclei identified with Hoechst in blue. Identification of glial cells with GFAP (D) and double-labeling with ADDLs (E). Overlay of ADDL positive and GFAP positive cell types (F) excludes ADDL binding to GFAP positive cells.

### DETAILED DESCRIPTION

Aβ (Abeta)-derived diffusible ligands (ADDLs) comprise the neurotoxic subset of Abeta 1-42 oligomers now implicated in synaptic malfunction and early stage memory loss en route to Alzheimer's disease (AD). Disruption in neuronal signaling and synaptic plasticity is caused by soluble Abeta (Ab) assemblies, rather than the fibrillar Ab deposited in plaques, suggesting a likely mode of action involving specific receptor-ligand interactions, rather than non-specific cellular damage.

Methods for the analysis of amyloid are known in the art (see e.g., U.S. Pat. No. 5,164,295; U.S. Pat. No. 5,223,482; U.S. Pat. No. 5,348,963; U.S. Pat. No. 5,547,841; U.S. Pat. No. 5,576,209; U.S. Pat. No. 5,652,092; U.S. Pat. No. 5,656,477; U.S. Pat. No. 5,693,478; U.S. Pat. No. 5,703,209; U.S. Pat. No. 5,721,106; U.S. Pat. No. 5,843,695; U.S. Pat. No. 6,001,331; U.S. Pat. No. 6,004,936; U.S. Pat. No. 6,194,163; U.S. Pat. No. 6,284,221; U.S. Pat. No. 6,294,340; U.S. Pat. No. 6,441,049; U.S. Pat. No. 6,518,011; U.S. Pat. No. 6,589,747; U.S. Pat. No. 6,600,947; U.S. Pat. No. 6,639,058; U.S. Pat. No. 6,677,299; U.S. Pat. Nos. 6,825,164; 6,949,575; and the like).

Additional methods are disclosed in U.S. Pat. No. 5,137,873; U.S. Pat. No. 5,200,339; U.S. Pat. No. 5,262,332; U.S. Pat. No. 5,434,050; U.S. Pat. No. 5,506,097; U.S. Pat. No. 5,514,653; U.S. Pat. No. 5,523,295; U.S. Pat. No. 5,538,845; U.S. Pat. No. 5,567,724; U.S. Pat. No. 5,622,981; U.S. Pat. No. 5,876,948; U.S. Pat. No. 5,958,964; U.S. Pat. No. 6,011,019; U.S. Pat. No. 6,107,050; U.S. Pat. No. 6,140,309; U.S. Pat. No. 6,210,655; U.S. Pat. No. 6,268,479; U.S. Pat. No. 6,274,119; U.S. Pat. No. 6,331,408; U.S. Pat. No. 6,413,512; U.S. Pat. No. 6,428,950; U.S. Pat. No. 6,555,651; U.S. Pat. No. 6,579,689; U.S. Pat. No. 6,649,346; U.S. Pat. No. 6,660,530; U.S. Pat. No. 6,737,038; U.S. Pat. No. 6,815,175; U.S. Pat. No. 6,878,363; and the like.

Disclosed herein is an image-based method for quantification of ADDL binding to primary hippocampal neurons in culture using the Cellomics ArrayScan imaging platform. Dissociated neurons from E18 rat hippocampus were cultured in 96-well microtiter plates for 16 days and exposed to increasing concentrations of ADDLs for 15 minutes, followed by fixation. ADDLs were visualized via immunohistochemistry utilizing an ADDL-specific monoclonal antibody, and neurons were identified using MAP2 immunostaining. Images were acquired at 10× magnification, and average ADDL binding intensity in MAP2 positive cells was measured in the proximal dendritic compartment.

ADDLs show selective binding to a sub-population of hippocampal neurons. Binding to individual neurons is heterogeneous and most prominent at higher concentrations suggesting a differential expression of ADDL binding sites or selective binding of ADDL species to particular neurons. Detection of ADDL binding is limited at low magnification, thus reducing the number of selected neurons at lower ADDL concentrations. This image analysis method is a valuable and quantitative tool for characterizing ADDL binding to synaptic receptors and evaluating molecules that block specific ADDL binding.

### EXAMPLE 1

### Primary Hippocampal Neuron Cultures.

Primary hippocampal cultures were prepared from embryonic day 18 (E18) rat brains. Cells were plated on 96-well microtiter plates, coated with poly-D-Lysine (50 mg/ml) at a density of 10,000/well. Hippocampal cultures were grown in Neurobasal medium supplemented with B27, 0.5 mM glutamine, 12.5 mM glutamate and penicillin/streptomycin. At 4DIV neurons were treated with AraC (2 mM) to inhibit glia proliferation.

### ADDL Labeling and Immunocytochemistry of Hippocampal Neurons.

ADDL were assembled according to Chromy et al., 2003. Live neurons (16 DIV) were incubated with increasing ADDL concentrations at 37° C. for 15 min. After washing with phosphate-buffered saline (PBS), the neurons were fixed for 15 min with 4% paraformaldehyde/4% sucrose in phosphate-buffered saline (PBS). After fixation cells were washed two times for 30 min at room temperature and incubated with primary antibodies 2B4 and MAP2 (Upstate) in buffer (2% BSA, 0.1% Triton X-100, 30 mM phosphate buffer pH 7.4) overnight at 4° C. Neurons were then washed three times in PBS for 30 min at room temperature and incubated with secondary antibody conjugated to Alexa 488 and Cy5 in buffer (2% BSA, 30 mM phosphate buffer pH7.4, 300 nM DAPI) for 2 hr at room temperature and washed three times in PBS for 30 min.

### Image Analysis and Quantification.

Images of labeled neurons were acquired and analyzed on a Cellomics ArrayScan® HCS Reader. Acquisition settings included imaging 10 fields per well at a 10× magnification. A proprietary modification of a Cellomics BioApplication was used for image analysis. Nuclei were identified using DAPI (Channel 1) and neurons were identified and selected for analysis by their staining by the MAP2 antibody (Channel2, CY5). The neuronal subpopulation was analyzed for ADDL binding in Channel 3 (FITC). BioApplication automatically reports the percentage of MAP2-labeled cells in each sample (well average of the 10 field) as well as level of ADDL binding in each individual cell. Images and numeric data were automatically transferred to Cellomics Store®, where well- and cell-level data were viewed for analysis. Cell level data were exported to Origin for Histogram analysis.

TABLE 1
Number of selected nuclei (DAPI)

| | ADDL Concentration | | | | |
|---|---|---|---|---|---|
| | 1 μM | 0.5 μM | 0.25 μM | 0.125 μM | No ADDL |
| Well A | 597 | 728 | 607 | 707 | 665 |
| Well B | 679 | 706 | 695 | 736 | 744 |
| Well C | 694 | 713 | 895 | 708 | 692 |
| Total | 1970 | 2147 | 2197 | 2151 | 2101 |

TABLE 2
Number of selected neurons (MAP2)

| | ADDL Concentration | | | | |
|---|---|---|---|---|---|
| | 1 μM | 0.5 μM | 0.25 μM | 0.125 μM | No ADDL |
| Well A | 564 | 705 | 587 | 671 | 625 |
| Well B | 643 | 663 | 663 | 692 | 713 |
| Well C | 652 | 674 | 823 | 667 | 662 |
| Total | 94 ± 0.3% | 94 ± 1.5% | 92 ± 2.5% | 94 ± 0.4% | 96 ± 1% |

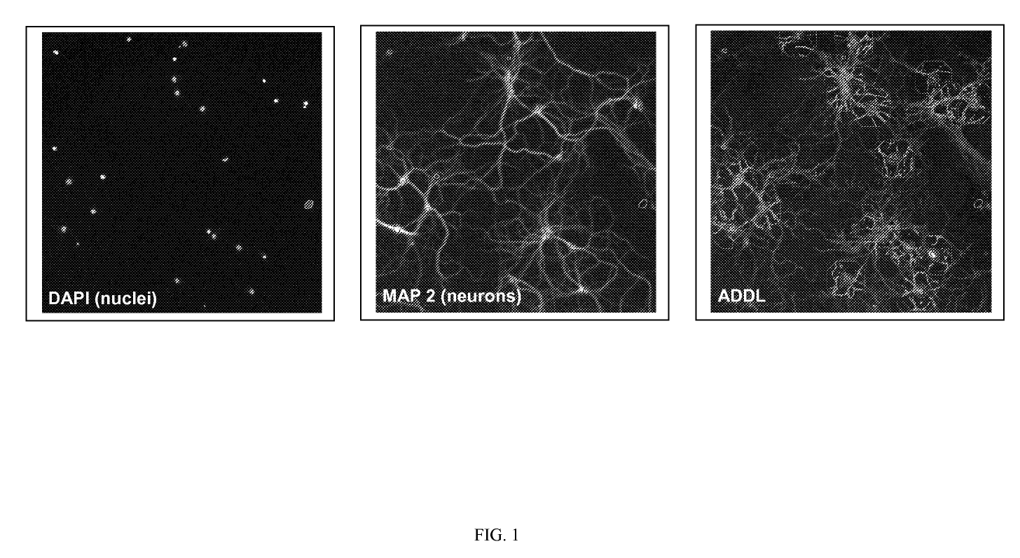

### Summary of Tables 1 & 2 (refer to FIG. 1):

Table 1: Number of selected nuclei identified in each well (n=3 for each concentration of ADDLs).

Table 2: Number of selected MAP2 positive cells in each well. The total neuronal population measured for each ADDL concentration is expressed as a percentage of nuclei identified. Non-neuronal cells are not included in the analysis.

TABLE 3
Summary table of ADDL positive neurons

| | ADDL Concentration | | | | |
|---|---|---|---|---|---|
| | 1 μM | 0.5 μM | 0.25 μM | 0.125 μM | No ADDL |
| No. of Cells | 1,859 | 2,042 | 2,073 | 2,030 | 2,000 |
| ADDL | 74% | 26.6% | 24% | 2.8% | 0% |
| positive | | | | | |
| ADDL | 26% | 73.4% | 76% | 97.2% | 100% |
| negative | | | | | |

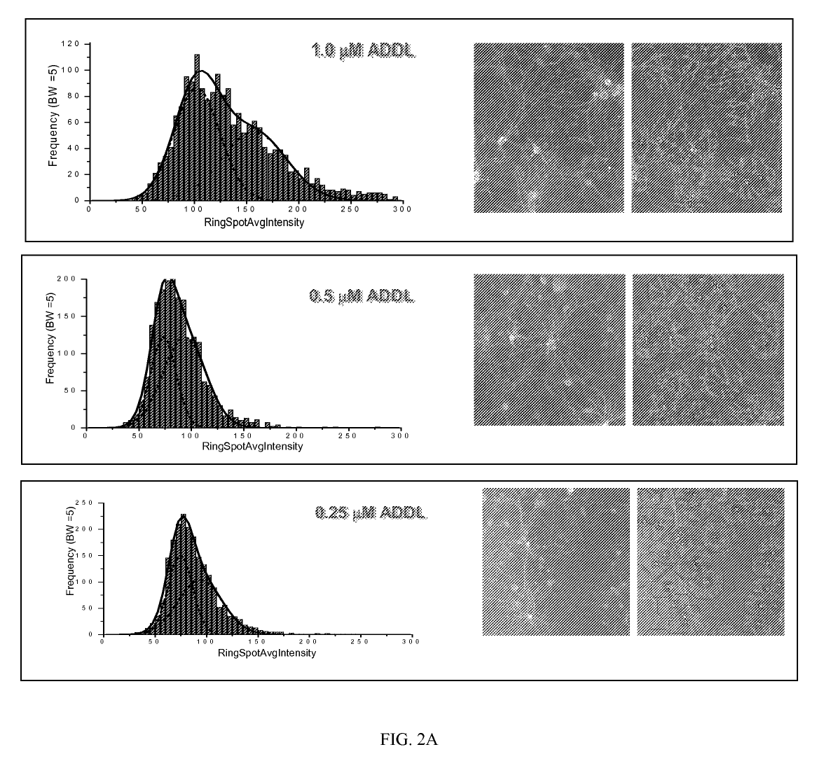

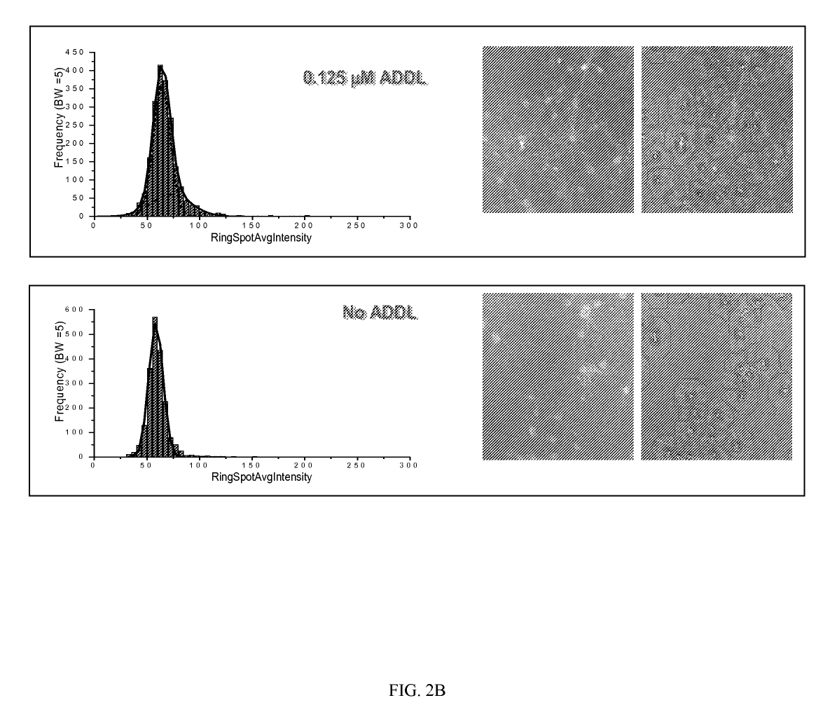

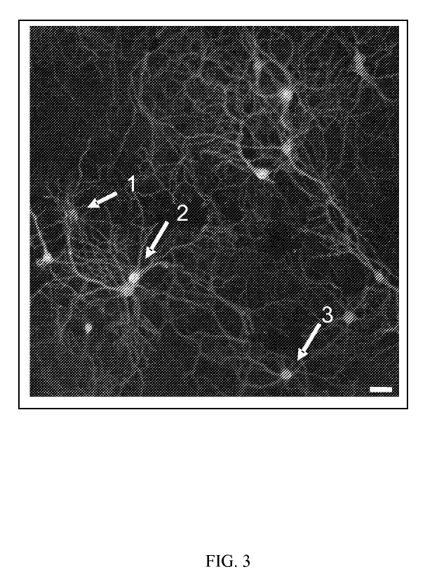

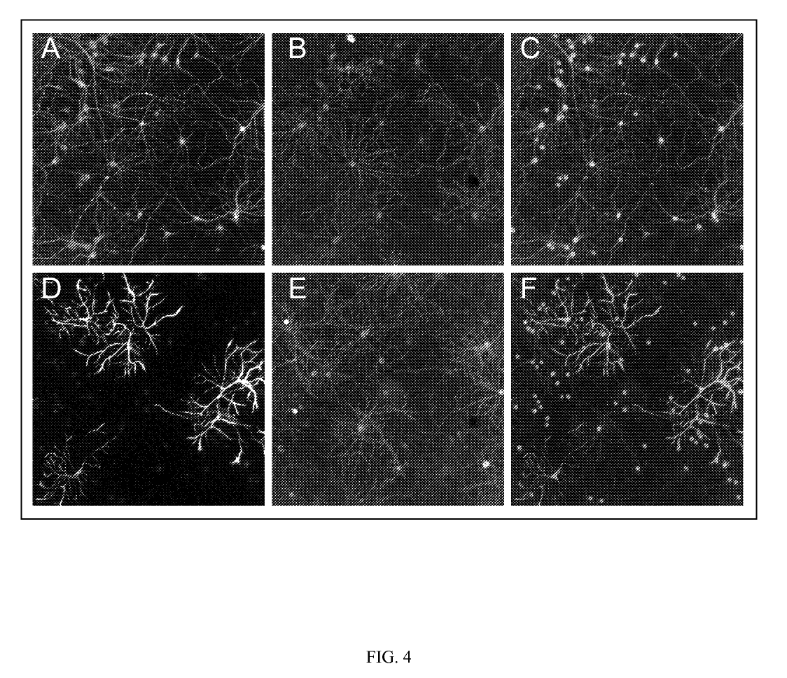

Table 3: Quantification of ADDL binding to hippocampal neurons in culture expressed as percentage of MAP2 positive cells at increasing ADDL concentrations. Note that the number of identified ADDL positive cells at low magnification is reduced with decreasing ADDL concentrations. (refer to FIGS. 2A, 2B, & 3)

This application is related to U.S. patent application Ser. No. 08/796,089, filed Feb. 5, 1997, now U.S. Pat. No. 6,218,506, issued Apr. 17, 2001; International Patent App. No. PCT/US98/02426, filed Feb. 5, 1998; U.S. patent application Ser. No. 09/369,236, filed Aug. 4, 1999, now U.S. patent application Ser. No. 11/100,212, filed Apr. 6, 2005; International Patent App. No. PCT/US00/21458, filed Aug. 4, 2000; U.S. patent application Ser. No. 09/745,057, filed Dec. 20, 2000, now U.S. patent application Ser. No. 11/130,566, filed May 16, 2005; U.S. patent application Ser. No. 10/166,856, filed Jun. 11, 2002; International Patent App. No. PCT/US03/19640, filed Jun. 11, 2003; U.S. patent application Ser. No. 10/676,871, filed Oct. 1, 2003, now U.S. patent application Ser. No. 10/924,372, filed Aug. 23, 2004, now U.S. patent application Ser. No. 11/142,869, filed Jun. 1, 2005; International Patent 15 App. No. PCT/US03/30930, filed Oct. 1, 2003; International Patent App. No. PCT/US05/17176, filed May 16, 2005; International Patent App. No. PCT/US05/23958, filed Jul. 5, 2005; and the like. All of the foregoing patents and patent applications are incorporated herein in their entirety by reference.

Unless otherwise noted, all patents, patent applications, as well as any other scientific and technical writings mentioned herein are incorporated by reference to the extent that they are not contradictory.

The preceding description of preferred embodiments is presented for purposes of illustration and description, and is not necessarily exhaustive nor intended to limit the claimed invention to the precise form(s) disclosed. The description was selected to best explain the principles of the invention and practical application of these principles to enable others skilled in the art to best utilize the claimed invention in various embodiments and with various modifications as are suited to the particular use contemplated. The scope of the claimed invention is not to be limited by the specification, but defined by the claims herein.

We claim:

1) A method for quantification of ADDL binding to neuronal cells to which ADDLs bind.

2) The method of claim 1, wherein the method identifies and selects neurons based on MAP2 immunoreactivity, thereby ensuring that glial cells are excluded from the ADDL binding analysis.

3) The method of claims 1 or 2, wherein ADDL binding occurs in a sub-population of 16 DIV neurons and is heterogeneous in intensity among individual cells.

4) The method of claims 1, 2, or 3, wherein the presence or absence of ADDL binding is used to evaluate molecules and antibodies that block ADDL binding.

5) A method of screening for ADDL binding inhibitors.

6) A method of screening for ADDL signalling.

7) A method of screening for cell types and receptors involved in ADDL signalling.

8) The method of claim 1 wherein the primary cultures of neurons are hippocampal cells.
